(19) United States
(12) Patent Application Publication
(10) Pub. No.: US 2005/0042262 A1
(43) Pub. Date: Feb. 24, 2005

(54) Effervescent cleansing article

(75) Inventors: Erik John Hasenoehrl, Loveland, OH (US); Kenneth Eugene Kyte III, Lebanon, OH (US); Dana Paul Gruenbacher, Fairfield, OH (US); Edward Dewey Smith III, Mason, OH (US)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/645,080

(22) Filed: Aug. 21, 2003

(51) Int. Cl. A61K 9/70

(52) U.S. Cl. 424/443

(57) ABSTRACT

The present invention relates to an article for cleansing body surfaces. The article contains an effervescent composition, a first layer and a cleansing composition. Surprisingly, the article provides improved lather and cleansing with a Steady Flash Lather Volume of 250 ml, a Steady Total Lather Volume of 850 ml and a Rinsability percent of greater than about 50%.

### CROSS-REFERENCE TO RELATED APPLICATIONS

This application claims priority to U.S. application Ser. No. 10/236,832, filed Sep. 6, 2002, which is a divisional of U.S. Pat. No. 6,495,151, granted Dec. 17, 2002, which is a divisional of U.S. Pat. No. 6,280,757, granted Aug. 28, 2001, which is a continuation-in-part (CIP) of U.S. Pat. No. 6,153,208, granted Nov. 28, 2000, which is a CIP of U.S. Pat. No. 6,190,678, granted Feb. 20, 2001, which is a CIP of U.S. Ser. No. 09/065,991, filed Apr. 24, 1998, now abandoned, which is a CIP of U.S. Pat. No. 6,132,746, granted Oct. 17, 2000.

### FIELD OF THE INVENTION

The invention relates to a cleansing article comprising an effervescent composition and a cleansing composition that has a Steady Flash Lather Volume of at least 250 ml, the article is useful for cleansing the skin and other keratinous regions.

### BACKGROUND OF THE INVENTION

Classically the process of cleansing skin or other sheets has employed a surfactant composition. Sometimes an implement has joined the composition. Articles such as articles or pads serve a multi-purpose including acting as delivery package for the surfactant. Articles may also assist in generating foam. They also function as an abrasive assisting in the cleansing function.

U.S. Pat. No. 4,234,442 (Cornelissens) describes an article that can consist of a water permeable material filled with an acidic and an alkaline constituent. Adipic, succinic and glutaric acids exemplify the acidic constituent. Sodium bicarbonate and carbonate represent the alkaline ingredient.

U.S. Pat. No. 4,272,393 (Gergely) describes a cleaning article formed of a porous flexible sheet, especially a cellulosic paper, impregnated with detergent and a gas-generating system. The latter is formed by separating an acidic component such as citric acid from a basic component such as sodium carbonate in two separate areas of the sheet.

U.S. Pat. No. 4,515,703 (Haq), U.S. Pat. No. 4,600,620 (Lloyd et al.), and U.S. Pat. No. 4,603,069 (Haq et al.) describe wiping articles impregnated with surfactant. These do not contain any effervescent ingredients.

WO 97/43366 (Askew et al.) reports an effervescent system to improve dispensability of granular laundry detergent powders into wash water of automatic washing machines. Citric acid and bicarbonate combinations are employed to generate effervescence.

WO 99/48469 (Yagnik et al.) discloses powdered compositions some of which may be delivered via a tea bag. These compositions are formulated with an exothermic ingredient, a pH adjusting agent such as citric acid, optionally sodium bicarbonate for an effervescent effect and an aromatic ingredient for fragrance.

U.S. Pat. Application 2003/0064042A1, (Bergquist), discloses envelope-like articles comprising effervescent cleansing composition capable of generating a foam upon contact with water; and a envelope formed of first and second water insoluble sheets, at least one being water permeable, the first and second sheets forming there between an area housing the cleansing composition, the second substrate comprising a non-woven sheet selected from the group consisting of meltblown, spunbond and sheet combinations.

U.S. Pat. Application 2003/0063136A1, (Bergquist), discloses a curvilinear envelope wherein the hydroentangled fibers are entangled in a direction perpendicular to a major longitudinal axis of the web; and an effervescent cleanser composition.

Common to those of the aforementioned systems is the employment of effervescent anhydrous powders within the article for generating gas and foam when immersed in water. The generation of gas by the effervescent powders creates by products which can inhibit the generation of and stability of a lather. Citric acid and sodium bicarbonate react to generate carbon dioxide gas but at the same time generate sodium citrate salt as a by product.

Accordingly, it is an object of the present invention to provide a cleansing article that is both effervescent and high lathering.

Still a further object of the invention is to provide a cleansing article that imparts a pleasant sensory feel to skin during and after use.

### SUMMARY OF THE INVENTION

The present invention relates to an article for cleansing body surfaces. The article comprises an effervescent composition, at least one layer and a cleansing composition. Surprisingly, the article provides improved lather and cleansing with a Steady Flash Lather Volume of 250 ml, a Steady Total Lather Volume of 850 ml, and a Rinsability percent greater than about 50%. It has been found that the effervescent composition when interspersed within a web of material within a pouch wherein the surfactant is located on the outer surfaces of the sheets making up said article provides for better foaming. In an embodiment of the present invention, the effervescent composition preferably comprising alkaline and acid materials that are applied into the web, the article of the present invention prevents unwanted aggregation or caking of the materials which interferes with the lather and foaming production of the article.

Also provided is a method for cleansing body surfaces involving wetting with water a cleansing article containing the effervescent composition and cleansing composition, generating foam and lather from the article and contacting skin surfaces with the generated foam/lather, particularly in the context of bathing. Further provided is a kit comprising said cosmetic articles in a package designed to prevent loss of the effervescence of said article during storage that can be resealed after initial use of the product thereby deterring premature activation of effervescence of the articles within said package.

### DETAILED DESCRIPTION OF THE INVENTION

By “composition associated with the “web”, “layer”, or “surface” as used herein, means compositions that are applied to or inside of the individual fibers prior to forming the web, layer or surface permeated into the web, layer or surface, coated onto, within or adjacent to the exposed surfaces of the web, layer or surface.

By a “lathering surfactant” is meant a surfactant, which when combined with a fluid and mechanically agitated generates a foam or lather.Layers

The article of the present invention is formed from a first layer that is comprised of a web. This web can be entirely made up of nonwovens, formed films, films, composite material, wovens and combinations and, or combination of nonwovens, formed films, films, composite material, wovens and combinations making up the web if desirable. In a preferred embodiment of the present invention the article is formed from a first layer further comprising a pouch that comprises the second layer of the article that is comprised of at least two webs. In another preferred embodiment of the present invention the article is formed from a first layer further comprising a laminate that comprises the second layer of the article that is comprised of at least two webs. The only requirement is that at least one of the layers be at least water-permeable to the extent that sufficient water passes through said layer for activating the effervescing composition located within the article formed by the layers.

Without being limited by theory, the layers can enhance cleansing. The layers can have the same or differing textures on each side. The layers may act as an efficient lathering and exfoliating implement. By physically coming into contact with the skin or hair, the layer significantly aids in cleansing and removal of dirt, makeup, dead skin, and other debris. In preferred personal care embodiments, however, the layers are non-scouring or nonabrasive to the skin.

Advantageously at least one of the layers should be sufficiently translucent to allow viewing from outside of the ingredients. A calendered pattern helps achieve a translucent panel while still retaining strength and water permeability. Articles may have any geometric shape including square, round, oval and tetrahedral configurations.

In a preferred embodiment the article has a third layer comprising a web that comprises a cleansing composition associated with at least one surface of the third layer. The third layer is in communication with and juxtaposed to the second layer.

In a preferred embodiment, the article has a fourth layer in communication with and juxtaposed to the third layer. The fourth layer comprises high loft material. As used herein, “loft” means that the layer has density of from about 0.00005 g/cm3 to about 0.1 g/cm3, preferably from about 0.001 g/cm3 to about 0.09 g/cm3 and a thickness of from about 0.1 mm to about 30 mm. Additionally, the high loft material of the fourth layer comprises a cleansing composition associated with the fourth layer.

The layers may comprise a variety of both natural and synthetic fibers or materials. As used herein, “natural fibers” are those derived from plants, animals, insects or byproducts of plants, animals, and insects. The conventional base starting material is usually a fibrous web comprising any of the common synthetic or natural textile-length fibers, or combinations thereof.

Nonlimiting examples of natural materials useful in the present invention include, but are not limited to, silk fibers, keratin fibers and cellulosic fibers. Nonlimiting examples of keratin fibers include those selected from the group consisting of wool fibers, camel hair fibers, and the like. Nonlimiting examples of cellulosic fibers include those selected from the group consisting of wood pulp fibers, cotton fibers, hemp fibers, jute fibers, flax fibers, Tencile® and combinations thereof. Cellulosic fiber materials are preferred in the present invention. Nonlimiting examples of synthetic materials useful in the present invention include those selected from the group consisting of acetate fibers, acrylic fibers, cellulose ester fibers, modacrylic fibers, polyamide fibers, polyester fibers, polyolefin fibers, polyvinyl alcohol fibers, rayon fibers, formed films, films, and combinations thereof. Examples of suitable synthetic materials include acrylics such as acrilan, creslan, and the acrylonitrile-based fiber, or Ion; cellulose ester fibers such as cellulose acetate, arnel, and acele; polyamides such as nylons (e.g., nylon 6, nylon 66, nylon 610, and the like); polyesters such as fortrel, kodel, and the polyethylene terephthalate fiber, polybutylene terephthalate fiber, dacron; polyolefins such as polypropylene, polyethylene; polyvinyl acetate fibers; polyurethane foams and combinations thereof. These and other suitable fibers and the nonwovens prepared there from are generally described in Riedel, “Nonwoven Bonding Methods and Materials,” Nonwoven World (1987); The Encyclopedia Americana, vol. 11, pp. 147-153, and vol. 26, pp. 566-581 (1984); U.S. Pat. No. 4,891,227, to Thaman et al., issued Jan. 2, 1990; and U.S. Pat. No. 4,891,228. As used herein, “nonwoven” means that the layer comprises fibers which are not woven into a fabric but rather are formed into a sheet, mat, or pad layer. The fibers can either be random (i.e., randomly aligned) or they can be carded (i.e., combed to be oriented in primarily one direction). Nonwoven substrates made from synthetic materials useful in the present invention can be obtained from a wide variety of commercial sources.

More preferred synthetic fibers for the layer are solid staple polyester fibers, which comprise polyethylene terephthalate homopolymers. Suitable synthetic materials may include solid single component and multicomponent synthetic fibers, i.e., more than one type of material making up the fibers. The synthetic fibers may comprise bicomponent or dual component fibers. Such bicomponent fibers may have a core and a sheath configuration or a side-by-side configuration. In either instance, the sheet layer may comprise either a combination of fibers comprising the above-listed materials or fibers which themselves comprise a combination of the above-listed materials.

In any instance, side-by side configuration or core-sheath configuration, the fibers of the layer may exhibit a helical or spiral configuration, particularly the bicomponent type fibers.

A preferred synthetic material for a scouring layer may comprise nylon fibers. A more preferred synthetic material comprises nylon fibers formed into a scrim layer having additional nylon fibers bonded thereto such that the additional fibers form arcs on the scrim layer.

Natural material nonwovens useful in the present invention may be obtained from a wide variety of commercial sources. Nonlimiting examples of suitable commercially available paper layers useful herein include Airtex®, an embossed airlaid cellulosic layer having a base weight of about 71 gsy, available from James River, Green Bay, Wis.; and Walkisoft®, an embossed airlaid cellulosic having a base weight of about 75 gsy, available from Walkisoft U.S.A., Mount Holly, N.C.

Additional suitable nonwoven materials include, but are not limited to, those disclosed in U.S. Pat. No. 4,447,294, issued to Osborn on May 8, 1984; U.S. Pat. No. 4,603,176 issued to Bjorkquist on Jul. 29, 1986; U.S. Pat. No. 4,981,557 issued to Bjorkquist on Jan. 1, 1991; U.S. Pat. No. 5,085,736 issued to Bjorkquist on Feb. 4, 1992; U.S. Pat. No. 5,138,002 issued to Bjorkquist on Aug. 8, 1992; U.S. Pat. No. 5,262,007 issued to Phan et al. on Nov. 16, 1993; U.S. Pat. No. 5,264,082, issued to Phan et al. on Nov. 23, 1993; U.S. Pat. No. 4,637,859 issued to Trokhan on Jan. 20, 1987; U.S. Pat. No. 4,529,480, issued to Trokhan on Jul. 16, 1985; U.S. Pat. No. 4,687,153 issued to McNeil on Aug. 18, 1987; U.S. Pat. No. 5,223,096, issued to Phan et al. on Jun. 29, 1993 and U.S. Pat. No. 5,679,222, issued to Rasch et al. on Oct. 21, 1997.

Additional suitable materials include but are not limited to, formed films and composite materials, i.e., multiply materials containing formed films. Preferably, such formed films comprise plastics which tend to be soft to the skin. Suitable soft plastic formed films include, but are not limited to, polyolefins such as low density polyethylenes (LDPE). Preferably, composites such as Vacuum Formed Laminates (VFL's), which are composites of fibrous nonwovens and formed films.

Additional formed films include microapertured 100 mesh film supplied by Tredegar, Inc., Terre Haute, Ind., USA 47808. Apertures can be formed in a film by any means such as by drawing a vacuum across the film; by forcing fluid such as water through the film while the film is supported in a fine screen such as a 100 mesh screen; by mechanical means such as punching, tearing, stretching; using energy such as heat or light. As used herein, “apertured” means that the layer includes well-defined openings. Well-defined openings are typically surrounded by well-defined land areas. Also, as used herein, “apertures” encompasses holes, perforations, cavities, and the like. The well-defined opening can be impermeable (as in a film, which would be a formed film or a perforated film, e.g.), or permeable. As used herein, “microapertured” generally refers to layers containing well-defined microscopic apertures (i.e., those not readily visible to the naked eye having 20/20 vision).

Methods of making nonwovens are well known in the art. Generally, these nonwovens can be made by air-laying, water-laying, meltblowing, coforming, spunbonding, or carding processes in which the fibers or filaments are first cut to desired lengths from long strands, passed into a water or air stream, and then deposited onto a screen through which the fiber-laden air or water is passed. The resulting layer, regardless of its method of production or composition, is then subjected to at least one of several types of bonding operations to anchor the individual fibers together to form a self-sustaining web. In the present invention the nonwoven layer can be prepared by a variety of processes including, but not limited to, meltblowing, spunbonding, air-entanglement, hydroentanglement, thermal bonding, and combinations of these processes.

Effervescent Composition

In the present invention a pouch that comprises the second layer of the article is comprised of at least two webs with the effervescent composition associated with the webs. In a preferred embodiment of the present invention a pouch that comprises the second layer of the article is comprised of at least two webs with the effervescent composition interposed between the webs. The webs hold the effervescent composition in such a way wherein after loading the web with such composition it does not agglomerate or spread unevenly throughout the interior of the article. This structure also prevents caking of the dry granular components making up the effervescing chemistry of the invention.

In another preferred embodiment of the present invention a laminate comprises the second layer that comprises at least two webs with the effervescent composition associated with the webs. The effervescent composition can also be interposed between the webs of the second layer.

The effervescent composition that is associated or interposed with the web disclosed above includes an acidic material. Suitable for this purpose are any acids present in dry solid form. Especially appropriate are C₂ to C₂₀ organic mono- and poly-carboxylic acids and especially alpha- and beta-hydroxycarboxylic acids; C₂ to C₂₀ organophosphorus acids such as phytic acid; C₂ to C₂₀ organosulfur acids such as toluene sulfonic acid; and peroxides such as hydrogen peroxide. Typical hydroxycarboxylic acids include adipic, glutaric, succinic, tartaric, malic, maleic, lactic, salicylic and citric acids as well as acid forming lactones such as gluconolactone and glucarolactone. Most preferred is citric acid. Also suitable as acid material may be encapsulated acids. Typical encapsulating material may include water-soluble synthetic or natural polymers such as polyacrylates (e.g. encapsulating polyacrylic acid), cellulosic gums, polyurethane and polyoxyalkylene polymers. By the term “acid” is meant any substance which when dissolved in deionized water at 1% concentration will have a pH of less than 7, preferably less than 6.5, optimally less than 5. These acids preferably at 25° C. are in solid form, i.e. having melting points no less than 25° C. Concentrations of the acid should range from about 0.5 to about 80%, preferably from about 10 to about 65%, more preferably from about 20 to about 45% by weight of the total composition.

Another component of the effervescent composition that is associated or interposed with the web disclosed above is that of an alkaline material. The alkaline material is a substance which can generate a gas such as carbon dioxide, nitrogen or oxygen, i.e. effervesce, when contacted with water and the acidic material. Suitable alkaline materials are anhydrous salts of carbonates and bicarbonates, alkaline peroxides (e.g. sodium perborate and sodium percarbonate) and azides (e.g. sodium azide). Preferably the alkaline material is sodium or potassium bicarbonate. Amounts of the alkaline material may range from about 1 to about 80%, preferably from about 5 to about 49%, more preferably from about 15 to about 40%, optimally from about 25 to about 35% by weight of the total composition.

By the term “anhydrous” is meant the presence of no more than 5%, preferably no more than 3.5% and optimally no more than 1% water by weight of the total composition. Water of hydration is not considered to be water for purposes of the anhydrous definition. However, it is preferred to minimize, preferably to eliminate any water of hydration.

Advantageously the combined amount of acidic and alkaline materials will be at least about 1.5%, preferably from about 40 to about 95%, optimally from about 60 to about 80% by weight of the total composition.

Cleansing Compositions

The present invention comprises cleansing articles. Preferably these articles are personal care articles with a personal care composition used by individuals primarily for cleansing and, or treatment of skin, hair or other and similar keratin-containing surfaces including skin, hair and finger and toe nails. Preferably, the articles of the present invention comprise one or more lathering surfactants that are associated with the article of the present invention. Thus the lathering or non-lathering surfactants can be added onto or impregnated into the article. Generally this will be done prior to the point of use of the article, i.e., the surfactants will be combined with the article before the article is ultimately wetted for use.

In the context of this application, lathering surfactant means a surfactant, that when combined with a fluid and mechanically agitated generates foam or lather sufficient to cause the article that it's associated with to form a lather. Preferably, these lathering surfactants and, or their combination with other surfactants should be mild, which means that these surfactants provide sufficient cleansing or detersive benefits but do not overly dry the skin or hair.

A wide variety of lathering surfactants are useful for the cleansing compositions described herein and include those selected from the group consisting of anionic lathering surfactants, nonionic lather surfactants, amphoteric lathering surfactants, hard water soluble surfactants and mixtures thereof. Non-limiting examples of lathering surfactants useful in the compositions of the present invention are disclosed in U.S. Pat. No. 6,280,757, to McAtee et al., issued Aug. 28, 2001. Generally, the lathering surfactants do not strongly interfere with deposition of any conditioning agents that are present, e.g., are fairly water soluble, and usually have an HLB value of above 10. Cationic surfactants can also be used as optional components, provided they do not negatively impact the overall lathering characteristics of the required lathering surfactants.

The cleansing compositions of the present invention comprise a sufficient amount of one or more lathering surfactants such that the compositions are capable of generating from at the least about 250 ml of Steady Flash Lather Volume more preferably at least about 275 ml of Steady Flash Lather Volume, even more preferably at least about 300 ml of Steady Flash Lather Volume, still even more preferably at least about 325 ml of Steady Flash Lather Volume, and even still more preferably at least 350 ml of Steady Flash Lather Volume according to the Steady Lather Volume Test described below. Preferably the cleansing composition generates at the least about 850 ml of Steady Total Lather Volume, preferably at least about 900 ml of Steady Total Lather Volume, more preferably at least about 950 ml of Steady Total Lather Volume, even more preferably at least about 1000 ml of Steady Total Lather Volume and still even more preferably at least about 1100 ml of Steady Total Lather Volume according to the Steady Lather Volume Test described below.

Generally the cleansing composition will preferably comprise no more than about 1600 weight percent by weight of the article of the lathering surfactant, preferably comprise no more than about 1000 weight percent, more preferably no more than about 800 weight percent, and still more preferably no more than about 600 weight percent by weight of the article of a lathering surfactant. Generally the cleansing composition will preferably comprise at least 15 weight percent by weight of the article of the lathering surfactant, preferably at least 25 weight percent, more preferably at least 50 weight percent, and still more preferably at least 60 weight percent by weight of the article of a lathering surfactant.

Suitable Surfactants for the personal care compositions described above include the following surfactants:

Anionic Lathering Surfactants

Non-limiting examples of anionic lathering surfactants useful in the compositions of the present invention are disclosed in McCutcheon's, Detergents and Emulsifiers, North American edition (1986), published by allured Publishing Corporation; McCutcheon's, Functional Materials, North American Edition (1992); and U.S. Pat. No. 3,929,678, to Laughlin et al., issued Dec. 30, 1975.

A wide variety of anionic lathering surfactants are useful herein. Non-limiting examples of anionic lathering surfactants include those selected from the group consisting of sarcosinates, sulfates, isethionates, taurates, phosphates, lactylates, glutamates, and mixtures thereof. Amongst the isethionates, the alkoyl isethionates are preferred, and amongst the sulfates, the alkyl and alkyl ether sulfates are preferred.

Other anionic materials useful herein include are fatty acid soaps (i.e., alkali metal salts, e.g., sodium or potassium salts) typically having from a fatty acid having about 8 to about 24 carbon atoms, preferably from about 10 to about 20 carbon atoms. These fatty acids used in making the soaps can be obtained from natural sources such as, for instance, plant or animal-derived glycerides (e.g., palm oil, coconut oil, soybean oil, castor oil, tallow, lard, etc.). The fatty acids can also be synthetically prepared. Soaps and their preparation are described in detail in U.S. Pat. No. 4,557,853.

Other anionic materials include phosphates such as monoalkyl, dialkyl, and trialkylphosphate salts. Non-limiting examples of preferred anionic lathering surfactants useful herein include those selected from the group consisting of sodium lauryl sulfate, ammonium lauryl sulfate, ammonium laureth sulfate, sodium laureth sulfate, sodium trideceth sulfate, ammonium cetyl sulfate, sodium cetyl sulfate, ammonium cocoyl isethionate, sodium lauroyl isethionate, sodium lauroyl lactylate, triethanolamine lauroyl lactylate, sodium caproyl lactylate, sodium lauroyl sarcosinate, sodium myristoyl sarcosinate, sodium cocoyl sarcosinate, sodium lauroyl methyl taurate, sodium cocoyl methyl taurate, sodium lauroyl glutamate, sodium myristoyl glutamate, and sodium cocoyl glutamate and mixtures thereof.

Especially preferred for use herein is ammonium lauryl sulfate, ammonium laureth sulfate, sodium lauroyl sarcosinate, sodium cocoyl sarcosinate, sodium myristoyl sarcosinate, sodium lauroyl lactylate, and triethanolamine lauroyl lactylate.

Non-Ionic Lathering Surfactants

Non-limiting examples of nonionic lathering surfactants for use in the compositions of the present invention are disclosed in McCutcheon's, Detergents and Emulsifiers, North American edition (1986), published by allured Publishing Corporation; and McCutcheon's, Functional Materials, North American Edition (1992);
Nonionic lathering surfactants useful herein include those selected from the group consisting of alkyl glucosides, alkyl polyglucosides, polyhydroxy fatty acid amides, alkoxylated fatty acid esters, lathering sucrose esters, amine oxides, and mixtures thereof.

Non-limiting examples of preferred nonionic surfactants for use herein are those selected form the group consisting of C₈-C₁₄ glucose amides, C₈-C₁₄ alkyl polyglucosides, sucrose cocoate, sucrose laurate, lauramine oxide, cocoamine oxide and mixtures thereof.

Amphoteric Lathering Surfactants

The term “amphoteric lathering surfactant,” as used herein, is also intended to encompass zwitterionic surfactants, which are well known to formulators skilled in the art as a subset of amphoteric surfactants.

A wide variety of amphoteric lathering surfactants can be used in the compositions of the present invention. Particularly useful are those which are broadly described as derivatives of aliphatic secondary and tertiary amines, preferably wherein the nitrogen is in a cationic state, in which the aliphatic radicals can be straight or branched chain and wherein one of the radicals contains an ionizable water solubilizing group, e.g., carboxy, sulfonate, sulfate, phosphate, or phosphonate.

Non-limiting examples of amphoteric surfactants useful in the compositions of the present invention are disclosed in McCutcheon's, Detergents and Emulsifiers, North American edition (1986), published by allured Publishing Corporation; and McCutcheon's, Functional Materials, North American Edition (1992).

Non-limiting examples zwitterionic surfactants are those selected from the group consisting of betaines, sultaines, hydroxysultaines, alkyliminoacetates, iminodialkanoates, aminoalkanoates, and mixtures thereof.

Preferred lathering surfactants for use herein are the following, wherein the anionic lathering surfactant is selected from the group consisting of ammonium lauroyl sarcosinate, sodium trideceth sulfate, sodium lauroyl sarcosinate, ammonium laureth sulfate, sodium laureth sulfate, ammonium lauryl sulfate, sodium lauryl sulfate, ammonium cocoyl isethionate, sodium cocoyl isethionate, sodium lauroyl isetlionate, sodium cetyl sulfate, sodium lauroyl lactylate, triethanolamine lauroyl lactylate, and mixtures thereof, wherein the non-ionic lathering surfactant is selected from the group consisting of lauramine oxide, cocoamine oxide, decyl polyglucose, lauryl polyglucose, sucrose cocoate, C₁₂₋₁₄ glucosamides, sucrose laurate, and mixtures thereof; and wherein the amphoteric lathering surfactant is selected from the group consisting of disodium lauroamphodiacetate, sodium lauroamphoacetate, cetyl dimethyl betaine, cocoamidopropyl betaine, cocoamidopropyl hydroxy sultaine, and mixtures thereof.

Mid-Chain Branched Surfactants

Mid-chain branched surfactants are described in detail in U.S. Pat. No. 6,335,312. Mid-chain branched surfactants are high lathering surfactants and are hard water tolerant. Typically, mid-chain branched surfactants are hard water soluble and have the corresponding structural formula: 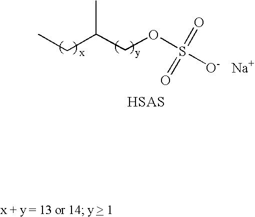 x+y=13 or 14; y≧1

An example of mid-chain branched surfactants includes ethoxylated mid-chain branched surfactants corresponding to the structural formula: 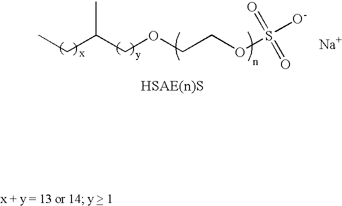 x+y=13 or 14; y≧1
Hard Water Tolerant Surfactants

Hard water tolerant surfactants are selected from the group consisting of nonionic lathering surfactants, phosphates, mid-chain branched surfactants and mixtures thereof.

Steady Lather Volume and Rinsability Tests

Articles of the present invention provide a steady lather profile as described hereafter. The Steady Lather Volume Test provides a measure of the lather profile of an article during its use cycle. The lather profile described herein is a combination of the Steady Flash Lather Volume and the Steady Total Lather Volume, both of which are determined in accordance with the Steady Lather Volume Test. Additionally, certain articles of the present invention have improved rinsability. It is desirable for disposable articles to exhaust their chemical components prior to disposal, in order that the article can be used for wiping as a final step, and in order to eliminate waste. Rinsability of articles is measured at the same time as lather volume is measured. Four 1,000 ml graduated cylinders are chosen which are marked in 10 ml increments and have a height of 14.5 inches at the 1,000 ml mark from the inside of the base (for example, Pyrex No. 2982). 100 grams of distilled water (+/−0.3 grams, at 23° C.) is added to each graduated cylinder. The cylinders, numbered sequentially from the first cylinder (Cylinder 1) to the last cylinder (Cylinder 4) are clamped in a rotating apparatus which clamps the cylinders with an axis of rotation which transects the center of the graduated cylinder in an axis parallel to the ground with the cylinder standing upright. Stopcocks are added to Cylinder 2 through Cylinder 4, the stopcocks being wound with Teflon tape to prevent leakage during the test if necessary.

The article is weighed and the weight recorded as the Article Initial Weight, Winitial. A small binder clip is attached to one edge of the article, the clip selected to be narrow enough to fit inside the neck of the graduated cylinders. A thin polymer thread such as a thin fishing line is tied to the clip so that when the thread is held aloft, the article hangs vertically from the clip and thread. The article is inserted into the first graduated cylinder (Cylinder 1) so that it hangs vertically inside the graduated cylinder with the top ends of the article hanging evenly with the 1,000 ml mark on the side of Cylinder 1. The stopcock is then inserted into the neck of Cylinder 1, fixing the article relative to the graduated cylinder.

The cylinders are automatically rotated 50 rotations by the rotating apparatus at a steady rate of 50 revolutions in 88 seconds in order to generate a lather, which is comprised of foam cells, and stopped in a vertical position to complete a first rotation sequence. After 15 seconds, the first lather volume, designated Lather in Cylinder 1, is measured to the nearest 10 ml mark by recording the lather height in ml up from the base in Cylinder 1 (said height includes water that has drained to the bottom, on top of which the lather is floating). If the top surface of the lather is uneven, the lowest height at which it is possible to see halfway across the first graduated cylinder is the Lather in Cylinder 1 (ml). If the lather is so coarse that a single or only a few foam cells reach across the entire cylinder, the height at which at least 5 foam cells are required to fill the diameter is the Lather in Cylinder 1, also in ml up from the base. Foam cells larger than one inch in diameter are designated as unfilled air instead of lather when they occur at the top surface of the lather. Lather that collects on the top of the graduated cylinder but does not move to the bottom of the graduated cylinder is also incorporated in the measurement if the lather on the top is in its own continuous layer, by adding the ml of foam collected there using a ruler to measure thickness of the layer and translating it to ml of volume, to the ml of lather measured up from the base. If a significant amount of lather (e.g., about 60 ml of volume or more) hangs on the side of the graduated cylinder making measurement of the total lather inaccurate or difficult, the lather on the top and sides is urged to the bottom part of the graduated cylinder to meet the other lather prior to measuring volume (during the 15 second drainage period) using a semi-circular shaped flexible plow attached to a rod, for example a 2 inch diameter plug cut from a sponge, cut again in half, and secured to a long threaded rod. The maximum lather volume possible is 1,000 ml (even if the total foam height exceeds the 1,000 ml mark on the graduated cylinder). The minimum lather volume possible is 100 ml (even if there is no foam, the height of the water in the graduated cylinder is designated the top of the lather and is measured as the Lather in Cylinder 1). After a total of 2 minutes of drainage time have elapsed, the thread is held and stopcock removed, and the article is removed from the first graduated cylinder, which is then re-cocked. Holding the article only by the thread, the article is fixed inside the second graduated cylinder, which is numbered as previously described as Cylinder 2, its stopcock is added, and a second rotation sequence is completed, the lather volume being measured in the second cylinder in the same manner as described for the first graduated cylinder, and is recorded as the Lather in Cylinder 2. This sequence is continued a total of four times, the lather volume in each graduated cylinder being recorded as the lather volume in that cylinder. That is, the lather volume in Cylinder 1, Cylinder 2, Cylinder 3, Cylinder 4 is designated, respectively, as the Lather in Cylinder 1, Lather in Cylinder 2, Lather in Cylinder 3, and Lather in Cylinder 4. The Steady Flash Lather Volume is the Lather in Cylinder 1. The Steady Total Lather Volume is obtained by adding together the lather obtained in all four graduated cylinders.

After 2 minutes of drainage time in the last graduated cylinder, the article is removed, unclipped, and placed in a tray in an oven at about 160° C. for at least 1 day, until the article is completely dry (article no longer loses weight over time if returned to oven). The dried weight of the article is recorded as the Article Dried Weight, W_{dried}. The article is then thoroughly rinsed with running hot water (greater than 55° C.) for several minutes, including slicing open to run water directly on inner layers of the laminate. When sliced, the article remains in one piece and no portions of the water insoluble article are lost during the rinsing. Any residual chemicals which are water soluble are completely exhausted by the rinsing. The article is returned to the same oven, dried in the same manner, and the dried, exhausted article is weighed and the weight recorded as the Article Substrate Weight, W_{substrate}. Rinsability in % is calculated from the weights, according to the following equation:
Rinsability=100%×[Wᵢₙᵢₜᵢₐₗ−W_{dried}]/[Wᵢₙᵢₜᵢₐₗ−W_{substrate}]
Certain articles of the present invention preferably have a Rinsability greater than about 50%, preferably greater than 55%, more preferably greater than 60%, even more preferably greater than 65%, still more preferably greater than 75%, and still even more preferably greater than 80%.

Optional Ingredients

The cleansing composition of the present invention may contain one or more additional skin care components. In a preferred embodiment, where the compositions are to be in contact with human keratinous tissue, the additional components should be suitable for application to keratinous tissue, that is, when incorporated into the composition they are suitable for use in contact with human keratinous tissue without undue toxicity, incompatibility, instability, allergic response, and the like within the scope of sound medical judgment.

The CTFA Cosmetic Ingredient Handbook, Second Edition (1992) describes a wide variety of nonlimiting cosmetic and pharmaceutical ingredients commonly used in the personal care industry, which are suitable for use in the compositions of the present invention.

In any embodiment of the present invention, however, the additional components useful herein can be categorized by the benefit they provide or by their postulated mode of action. However, it is to be understood that the additional components useful herein can in some instances provide more than one benefit or operate via more than one mode of action. Therefore, classifications herein are made for the sake of convenience and are not intended to limit the active to that particular application or applications listed.

Benefit Agents

The articles of the present invention can comprise a benefit agent that is useful for providing a therapeutic benefit and/or cosmetic benefit to the skin, hair and similar keratin-containing surfaces during the use of the article. The benefit agents are suitable for application to keratin-containing tissue, that is, they are suitable for use in contact with human keratin-containing tissue without undue toxicity, incompatibility, instability, allergic response, and the like within the scope of sound medical judgment.

The benefit agent can comprise no more than about 1600 weight percent, preferably no more than about 1000 weight percent of a substrate, more preferably no more than about 800 weight percent, and most preferably no more than about 600 weight percent of a skin treatment agent. The benefit agent can comprise at least 0.05 weight percent of a substrate, preferably at least 15 weight percent, more preferably at least 15 weight percent, and most preferably no more than about 60 weight percent of a substrate of a benefit agent.

The benefit agents useful in the present invention can comprise compositions described herein. The optional components useful herein can be categorized by their therapeutic or aesthetic benefit or their postulated mode of action. However, it is to be understood that the optional components useful herein can in some instances provide more than one therapeutic or aesthetic benefit or operate via more than one mode of action. Therefore, classifications herein are made for the sake of convenience and are not intended to limit the component to that particular application or applications listed. Also, when applicable, the pharmaceutically-acceptable salts of the components are useful herein.

The articles of the present invention may optionally contain one or more of such optional ingredients. Examples of these ingredient classes include: enzymes, abrasives, skin exfoliating agents, absorbents, aesthetic components such as fragrances, pigments, colorings/colorants, essential oils, skin sensates, astringents, etc. (e.g., clove oil, menthol, camphor, eucalyptus oil, eugenol, menthyl lactate, witch hazel distillate), anti-acne agents (e.g., resorcinol, sulfur, salicylic acid, erythromycin, zinc, etc.), anti-caking agents, antifoaming agents, additional antimicrobial agents (e.g., iodopropyl butylcarbamate), antioxidants, binders, biological additives, buffering agents, bulking agents, chelating agents, chemical additives, colorants, cosmetic astringents, cosmetic biocides, denaturants, drug astringents, external analgesics, film formers or materials, e.g., polymers, for aiding the film-forming properties and substantivity of the composition (e.g., copolymer of eicosene and vinyl pyrrolidone), humectants, opacifying agents, pH adjusters, propellants, reducing agents, sequestrants, skin bleaching agents (or lightening agents) (e.g., hydroquinone, kojic acid, ascorbic acid, magnesium ascorbyl phosphate, ascorbyl glucosamine), skin-conditioning agents (humectants, including miscellaneous and occlusive), skin soothing and/or healing agents (e.g., panthenol and derivatives (e.g., ethyl panthenol), aloe vera, pantothenic acid and its derivatives, allantoin, bisabolol, and dipotassium glycyrrhizinate), skin treating agents, including agents for preventing, retarding, arresting, and/or reversing skin wrinkles (e.g., alpha-hydroxy acids such as lactic acid and glycolic acid and beta-hydroxy acids such as salicylic acid), thickeners, hydrocolloids, particular zeolites, and vitamins and derivatives thereof (e.g. tocopherol, tocopherol acetate, beta carotene, retinoic acid, retinol, retinoids, retinyl palmitate, niacin, niacinamide, and the like). The articles of the present invention may include carrier components such as are known in the art. Such carriers can include one or more compatible liquid or solid filler diluents or vehicles which are suitable for application to skin or hair.

The articles of the present invention may optionally contain one or more of such optional components. Preferred articles optionally contain a safe and effective amount of an therapeutic benefit component comprising a therapeutic benefit agent selected from the group consisting of vitamin compounds, conditioning agents, skin treating agents, anti-acne actives, anti-wrinkle actives, anti-skin atrophy actives, anti-inflammatory actives, topical anesthetics, artificial tanning actives and accelerators, anti-microbial actives, anti-fungal actives, sunscreen actives, anti-oxidants, skin exfoliating agents, and combinations thereof. As used herein, “a safe and effective amount” means an amount of a compound or component sufficient to significantly induce a positive effect or benefit, but low enough to avoid serious side effects, (e.g., undue toxicity or allergic reaction), i.e., to provide a reasonable benefit to risk ratio, within the scope of sound medical judgment.

Conditioning Agents

The articles of the present invention can comprise a conditioning agent that is useful for providing a conditioning benefit to the skin, hair and other parts of the body with keratin-containing tissue. The conditioning agent can comprise no more than about 1600 weight percent of a substrate, preferably no more than about 1000 weight percent, more preferably no more than about 800 weight percent, and most preferably no more than about 600 weight percent of a conditioning agent. The conditioning agent can comprise at least 0.05 weight percent of a substrate, preferably at least 15 weight percent, more preferably at least 15 weight percent, and most preferably no more than about 60 weight percent of a conditioning agent.

The conditioning agent useful in the present invention can comprise: a water soluble conditioning agent or an oil soluble conditioning agent. The water soluble conditioning agent and oil soluble conditioning agent can be combined to form a conditioning emulsion. The oil soluble conditioning agent is selected from one or more oil soluble conditioning agents such that the weighted arithmetic mean solubility parameter of the oil soluble conditioning agent is less than or equal to 10.5.

Solubility parameters are well known to the formulation chemist of ordinary skill in the art and are routinely used as a guide for determining compatibilities and solubilities of materials in the formulation process. See “Solubility Effects in Product, Package, Penetration, and Preservation”, Cosmetics and Toiletries vol. 103, p 47-69, (October 1988).

Non-limiting examples of useful conditioning agents include those selected from the group consisting of petrolatum, fatty acids, esters of fatty acids, fatty alcohols, ethoxylated alcohols, polyol polyesters, glycerine, glycerin mono-esters, glycerin polyesters, epidermal and sebaceous hydrocarbons, lanolin, straight and branched hydrocarbons, silicone oil, silicone gum, vegetable oil, vegetable oil adduct, hydrogenated vegetable oils, nonionic polymers, natural waxes, synthetic waxes, polyolefinic glycols, polyolefinic monoester, polyolefinic polyesters, cholesterols, cholesterol esters, triglycerides and mixtures thereof.

More particularly, the conditioning agent may be selected from the group consisting of paraffin, mineral oil, petrolatum, stearyl alcohol, cetyl alchohol, cetearyl alcohol, behenyl alcohol, C10⁻³⁰ polyesters of sucrose, stearic acid, palmitic acid, behenic acid, oleic acid, linoleic acid, myristic acid, lauric acid, ricinoleic acid, steareth-1-100, cetereath 1-100, cholesterols, cholesterol esters, glyceryl tribehenate, glyceryl dipalmitate, glyceryl monostearate, trihydroxystearin, ozokerite wax, jojoba wax, lanolin wax, ethylene glycol distearate, candelilla wax, carnauba wax, beeswax, and silicone waxes.

Mineral oil, which is also known as petrolatum liquid, is a mixture of liquid hydrocarbons obtained from petroleum. See The Merck Index, Tenth Edition, Entry 7048, p. 1033 (1983) and International Cosmetic Ingredient Dictionary, Fifth Edition, vol. 1, p.415-417 (1993).

Petrolatum, which is also known as petroleum jelly, is a colloidal system comprising nonstraight-chain solid hydrocarbons and high-boiling liquid hydrocarbons. See The Merck Index, Tenth Edition, Entry 7047, p. 1033 (1983); Schindler, Drug. Cosmet. Ind., 89, 36-37, 76, 78-80, 82 (1961); and International Cosmetic Ingredient Dictionary, Fifth Edition, vol. 1, p. 537 (1993).

Nonvolatile silicones such as polydialkylsiloxanes, polydiarylsiloxanes, and polyalkarylsiloxanes are also useful skin conditioning agents. These silicones are disclosed in U.S. Pat. No. 5,069,897, to Orr, issued Dec. 3, 1991.

Cationic Polymers

The present invention may also contain an organic cationic deposition polymer. Concentrations of the cationic deposition polymer preferably range from about 0.025% to about 3%, more preferably from about 0.05% to about 2%, even more preferably from about 0.1% to about 1%, by weight of the cleansing composition.

Suitable cationic deposition polymers for use in the present invention contain cationic nitrogen-containing moieties such as quaternary ammonium or cationic protonated amino moieties. The cationic protonated amines can be primary, secondary, or tertiary amines (preferably secondary or tertiary), depending upon the particular species and the selected pH of the personal cleansing composition. The average molecular weight of the cationic deposition polymer is between about 5,000 to about 10 million, preferably at least about 100,000, more preferably at least about 200,000, but preferably not more than about 2 million, more preferably not more than about 1.5 million. The polymers also have a cationic charge density ranging from about 0.2 meq/gm to about 5 meq/gm, preferably at least about 0.4 meq/gm, more preferably at least about 0.6 meq/gm., at the pH of intended use of the personal cleansing composition, which pH will generally range from about pH 4 to about pH 9, preferably between about pH 5 and about pH 8.

Nonlimiting examples of cationic deposition polymers for use in the personal care composition include polysaccharide polymers, such as cationic cellulose derivatives. Preferred cationic cellulose polymers are the salts of hydroxyethyl cellulose reacted with trimethyl ammonium substituted epoxide, referred to in the industry (CTFA) as Polyquatemium 10 which are available from Amerchol Corp. (Edison, N.J., USA) in their Polymer KG, JR and LR series of polymers with the most preferred being KG-30M.

Other suitable cationic deposition polymers include cationic guar gum derivatives, such as guar hydroxypropyltrimonium chloride, specific examples of which include the Jaguar series (preferably Jaguar C-17) commercially available from Rhodia Inc., and N-Hance polymer series commercially available from Aqualon.

Other suitable cationic deposition polymers include synthetic cationic polymers. The cationic polymers suitable for use in the cleansing composition herein are water soluble or dispersible, non crosslinked, cationic polymers having a cationic charge density of from about 4 meq/gm to about 7 meq/gm, preferably from about 4 meq/gm to about 6 meq/gm, more preferably from about 4.2 meq/gm to about 5.5 meq/gm. The select polymers also must have an average molecular weight of from about 1,000 to about 1 million, preferably from about 10,000 to about 500,000, more preferably from about 75,000 to about 250,000.

The concentration of the cationic polymer in the personal care composition ranges from about 0.025% to about 5%, preferably from about 0.1% to about 3%, more preferably from about 0.2% to about 1%, by weight of the composition.

A nonlimiting example of a commercially available synthetic cationic polymer for use in the cleansing composition is polymethyacrylamidopropyl trimonium chloride, available under the trade name POLYCARE 133, from Rhodia, Cranberry, N.J., U.S.A.

Cationic Surfactants

Cationic surfactants are typically categorized as non-lathering surfactants but may be used in the articles of the present invention provided they do not negatively impact the desired benefits of the articles.

Nonlimiting examples of cationic surfactants useful herein are disclosed in McCutcheon's, Detergents and Emulsifiers, North American edition (1986), published by allured Publishing Corporation; and McCutcheon's, Functional Materials, North American Edition (1992); both of which are incorporated by reference herein in their entirety.

Nonlimiting examples of cationic surfactants useful herein include cationic alkyl ammonium salts such as those having the formula:
R₁R₂R₃R₄N⁺X⁻
wherein R₁, is selected from an alkyl group having from about 12 to about 18 carbon atoms, or aromatic, aryl or alkaryl groups having from about 12 to about 18 carbon atoms; R₂, R₃, and R₄ are independently selected from hydrogen, an alkyl group having from about 1 to about 18 carbon atoms, or aromatic, aryl or alkaryl groups having from about 12 to about 18 carbon atoms; and X is an anion selected from chloride, bromide, iodide, acetate, phosphate, nitrate, sulfate, methyl sulfate, ethyl sulfate, tosylate, lactate, citrate, glycolate, and mixtures thereof. Additionally, the alkyl groups can also contain ether linkages, or hydroxy or amino group substituents (e.g., the alkyl groups can contain polyethylene glycol and polypropylene glycol moieties).

More preferably, R₁ is an alkyl group having from about 12 to about 18 carbon atoms; R₂ is selected from H or an alkyl group having from about 1 to about 18 carbon atoms; R₃ and R₄ are independently selected from H or an alkyl group having from about 1 to about 3 carbon atoms; and X is as described in the previous paragraph.

Most preferably, R₁ is an alkyl group having from about 12 to about 18 carbon atoms; R₂, R₃, and R₄ are selected from H or an alkyl group having from about 1 to about 3 carbon atoms; and X is as described previously.

Alternatively, other useful cationic surfactants include amino-amides, wherein in the above structure R₁ is alternatively R₅CO—(CH₂)ₙ—, wherein R₅ is an alkyl group having from about 12 to about 22 carbon atoms, and n is an integer from about 2 to about 6, more preferably from about 2 to about 4, and most preferably from about 2 to about 3. Nonlimiting examples of these cationic emulsifiers include stearamidopropyl PG-dimonium chloride phosphate, stearamidopropyl ethyldimonium ethosulfate, stearamidopropyl dimethyl (myristyl acetate) ammonium chloride, stearamidopropyl dimethyl cetearyl ammonium tosylate, stearamidopropyl dimethyl ammonium chloride, stearamidopropyl dimethyl ammonium lactate, and mixtures thereof.

Nonlimiting examples of quaternary ammonium salt cationic surfactants include those selected from the group consisting of cetyl ammonium chloride, cetyl ammonium bromide, lauryl ammonium chloride, lauryl ammonium bromide, stearyl ammonium chloride, stearyl ammonium bromide, cetyl dimethyl ammonium chloride, cetyl dimethyl ammonium bromide, lauryl dimethyl ammonium chloride, lauryl dimethyl ammonium bromide, stearyl dimethyl ammonium chloride, stearyl dimethyl ammonium bromide, cetyl trimethyl ammonium chloride, cetyl trimethyl ammonium bromide, lauryl trimethyl ammonium chloride, lauryl trimethyl ammonium bromide, stearyl trimethyl ammonium chloride, stearyl trimethyl ammonium bromide, lauryl dimethyl ammonium chloride, stearyl dimethyl cetyl ditallow dimethyl ammonium chloride, dicetyl ammonium chloride, dicetyl ammonium bromide, dilauryl ammonium chloride, dilauryl ammonium bromide, distearyl ammonium chloride, distearyl ammonium bromide, dicetyl methyl ammonium chloride, dicetyl methyl ammonium bromide, dilauryl methyl ammonium chloride, dilauryl methyl ammonium bromide, distearyl methyl ammonium chloride, distearyl dimethyl ammonium chloride, distearyl methyl ammonium bromide, and mixtures thereof. Additional quaternary ammonium salts include those wherein the C12 to C22 alkyl carbon chain is derived from a tallow fatty acid or from a coconut fatty acid. The term “tallow” refers to an alkyl group derived from tallow fatty acids (usually hydrogenated tallow fatty acids), which generally have mixtures of alkyl chains in the C16 to C18 range. The term “coconut” refers to an alkyl group derived from a coconut fatty acid, which generally have mixtures of alkyl chains in the C12 to C14 range. Examples of quaternary ammonium salts derived from these tallow and coconut sources include ditallow dimethyl ammonium chloride, ditallow dimethyl ammonium methyl sulfate, di(hydrogenated tallow) dimethyl ammonium chloride, di(hydrogenated tallow) dimethyl ammonium acetate, ditallow dipropyl ammonium phosphate, ditallow dimethyl ammonium nitrate, di(coconutalkyl)dimethyl ammonium chloride, di(coconutalkyl)dimethyl ammonium bromide, tallow ammonium chloride, coconut ammonium chloride, stearamidopropyl PG-dimonium chloride phosphate, stearamidopropyl ethyldimonium ethosulfate, stearamidopropyl dimethyl (myristyl acetate) ammonium chloride, stearamidopropyl dimethyl cetearyl ammonium tosylate, stearamidopropyl dimethyl ammonium chloride, stearamidopropyl dimethyl ammonium lactate, and mixtures thereof.

Preferred cationic surfactants useful herein include those selected from the group consisting of dilauryl dimethyl ammonium chloride, distearyl dimethyl ammonium chloride, dimyristyl dimethyl ammonium chloride, dipalmityl dimethyl ammonium chloride, distearyl dimethyl ammonium chloride, and mixtures thereof.

Manufacturing the Articles

Articles according to the present invention may be formed in a variety of ways. An illustrative but certainly non-limiting example is as follows. Constituents of the effervescent composition (phase C in examples below) is placed into a dry mill or similar apparatus with a binder material and blended until a uniformly distributed powder results. Articles are formed of water permeable laminates whereby the effervescent composition is placed onto the first layer and subsequently a second layer is placed on top of said first layer. The two layers proceed through a heated lamination nip to bond the layers together. Subsequently, the effervescent composition/layer is unwound and laminated to the other layers by ultrasonic bonding.

Heat bonding may be employed as an alternative to ultrasonic bonding. Thread stitching, glue application or other closure mechanisms may also be utilized.

Liquid fragrance is sprayed onto any of the nonwoven layers (except Batting layer). Except in the operating and comparative examples, or where otherwise explicitly indicated, all numbers in this description indicating amounts of material are to be understood as modified by the word “about”.

The term “comprising” is meant not to be limiting to any subsequently stated elements but rather to encompass non-specified elements of major or minor functional importance. In other words the listed steps, elements or options need not be exhaustive.

All measurements referred to herein are made at 25° C., unless otherwise specified. All publications, patent applications and issued patents mentioned herein are hereby incorporated in their entirety by reference. Citation of any reference is not an admission regarding any determination as to its availability as prior art to the claimed invention.

Upon manufacture of the articles described above, a plurality of the articles are inserted into packaging and is sealed. The package also has a closure that can be repeatedly opened and closed to gain access to the supply of articles in the package. Such packaging is disclosed in U.S. Pat. No. 5,050,763, to Christensson, issued Sep. 24, 1999. After purchase of the product, the consumer opens the closure, removes the seal and removes a desired number of the cleansing articles found therein. The consumer closes the closure for storing the remaining non-selected articles until their next use wherein the closure is again opened to provide access to the articles in the package. Ideally, the closure is one that seals the package even after removing the seal upon the initial use of the article. This way the articles are protected from moisture and humidity sufficiently enough to prevent unwanted reactivity of the effervescent composition of the article prior to use. Furthermore, the package may comprise vents to allow escape of any residual gas from within the sealed package.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention. All exemplified amounts are concentrations by weight of the total cleansing, treatment compositions, unless otherwise specified.

### Example 1

An effervescent cleanser product can be prepared using the following ingredients. Phase A can be mixed with simple mixing equipment and coated using a slot coater on to the topside of Nonwoven layer 2 at a rate of 12 grams/sq meter. Phase A is also can be coated using a slot coater to the bottom of layer 4 at a rate of 12 grams/sq meter. Phase B can be added to the bottom side of layer 4 at a rate of 3.5 grams/sq meter. Phase C can be dry blended in an auger and laminated into place at a rate of 390 grams.square meter.

| Ingredient | Weight % |
|---|---|
| Phase A(@ 12 | |
| gsm × 2 layers) | |
| Glycerin | 2.21 |
| Tegobetaine FB | 1.46 |
| Plantaren 2000 N UP | 1.46 |
| Hamposyl L-30 | 1.46 |
| Butylene Glycol | 0.44 |
| Polyox WSR N3000 | 0.22 |
| Ucare Polymer JR30M | 0.11 |
| D-Panthenol | 0.089 |
| Emeressence 1160 | 0.066 |
| Benzyl Alcohol NF | 0.066 |
| Methyl Paraben | 0.055 |
| Propyl Paraben | 0.033 |
| Disodium EDTA | 0.021 |
| Grape Seed Extract | 0.0024 |
| Chamomile Extract | 0.0024 |
| Veragel 1:1 | 0.0024 |
| Vitamin E Acetate | 0.0024 |
| Phase B | |
| Super White Protopet | 0.81 |
| Phase C | |
| Sodium Bicarbonate | 50.33 |
| Citric Acid | 41.16 |
| Nonwoven Material | |
| Layer 1 | High Loft Batting (84 gsm, 40% |
| Layer 2 | bicomponent PET/PE |
| Layer 3 | core/sheath fiber, 2 mm thick) |
| Layer 4 | HET (hydroentangled)(50 gsm, |
| | 50% PP, 50% Tencel ®) |
| | Powder incorporated web |
| | Textured HET(60 gsm, |
| | 50% PP, 50% Tencel ®) |

### Example 2

An effervescent cleanser product can be prepared using the following ingredients. Phase A can be mixed with simple mixing equipment and coated using a slot coater on to the topside of Nonwoven layer 1 at a rate of 12 grams/sq meter. Phase A can also be coated using a slot coater to the bottom of layer 3 at a rate of 12 grams/sq meter. Phase B is added to the bottom side of layer 3 at a rate of 3.5 grams/sq meter. Phase C was dry blended in an auger and laminated into place at a rate of 390 grams.square meter.

| Ingredient | Weight % |
|---|---|
| Phase A(@ 12 | |
| gsm × 2 layers) | |
| Glycerin | 2.21 |
| Hydroxysultaine | 1.46 |
| Plantaren 2000 N UP | 1.46 |
| Hamposyl L-30 | 1.46 |
| Butylene Glycol | 0.44 |
| Polyox WSR N3000 | 0.22 |
| Ucare Polymer JR30M | 0.11 |
| D-Panthenol | 0.089 |
| Emeressence 1160 | 0.066 |
| Benzyl Alcohol NF | 0.066 |
| Methyl Paraben | 0.055 |
| Propyl Paraben | 0.033 |
| Disodium EDTA | 0.021 |
| Grape Seed Extract | 0.0024 |
| Chamomile Extract | 0.0024 |
| Veragel 1:1 | 0.0024 |
| Vitamin E Acetate | 0.0024 |
| Fragrance | 0.0024 |
| Phase B | |
| Super White Protopet | 0.81 |
| Phase C | |
| Sodium Bicarbonate | 50.33 |
| Citric Acid | 41.16 |
| Nonwoven Material | |
| Layer 1 | Polypropylene Spunbond (50 gsm) |
| Layer 2 | Powder incorporated web, HET(50 gsm, |
| Layer 3 | 50% PP, 50% Tencel ®) |
| | Textured HET(60 gsm, 50% PP, 50% Tencel ®) |

### Example 3

An article can be prepared for cleansing the body. A lathering surfactant component can be prepared using the following ingredients. The ingredients are prepared by mixing the cationic polymer with the glycol and surfactants under heat with continuous stirring to avoid lumps. Foaming is avoided. The perfume is added during cooling.

| | | Amount of |
|---|---|---|
| | | Ingredient |
| Ingredient | Supplier or common CTFA name | added |
| Alkyl Glyceryl | (Procter & Gamble Co., Iowa City, | 62.8% |
| Sulfonate (AGS) | Iowa, USA) | |
| 47.5% solids | | |
| paste | | |
| Cocamidopropyl | (Stepan Chemical) AMPHOSOL | 19.7% |
| Betaine, | CG | |
| 30% active | | |
| Citric Acid | Citric acid | 0.2% |
| Anhydrous | | |
| Propylene | Propylene glycol | 15.2% |
| Glycol | | |
| Polyox | (Amerchol) PEG 90M | 0.20% |
| WSR-301 | | |
| N-Hance | (Aqualon-Hercules, Irvine, CA, | 0.50% |
| 3196 | USA) cationic guar or guar | |
| | hydroxypropyltrimonium chloride | |
| | (polymer) | |
| Perfume | | 1.0% |
| Preservative | | 0.4% |
| & misc. | | |

The first side comprises a layer of a batting (84 gsm, 50% bicomponent fiber, 2 mm thick at 30-40 gsi). 5.0 grams of the surfactant component is added to the batting around the edges, avoiding the center area. A second layer which is a microapertured formed film having hydroformed apertures measuring about 100 microns by 200 microns is placed on the batting, male side facing away from the batting (Tredegar Inc., Terre Haute, Ind., USA). 2.1 grams of citric acid and 2.9 grams of sodium bicarbonate are added onto the formed film in the center area. A layer of 1 mil thick polyethylene film is placed on top of the chemicals. A layer of a soft, 70% rayon/30% bicomponent spunlace nonwoven (60 gsm) is placed as the final layer. The article is sealed using a pattern of S-shaped dots which measure about 8 mm long×2 mm wide, the long dimension parallel to the MD, and which are in rows spaced 16 mm apart. The dots are spaced 16 mm center to center within rows. The seal is effected by an ultrasonic sealer (Branson 921) or a thermal bonder (Sencorp Sealer at 315° F. for 111 seconds). A continuous perimeter seal is effected using a Vertrod electrical impulse bar sealer and the article is cut leaving an unsealed portion outside the perimeter. The article measures 11.6 cm×13.4 cm. The article is laid with the surfactant stripes up to dry at 120° F. and less than 5% humidity for 48 hours. The article is packaged in a hermetically sealed container until use. During use in the shower, the article lathers profusely for several minutes, and billows to a thickness of about 1.8 to 2.0 cm, maintaining its thickness for the duration one shower.

All documents cited in the Detailed Description of the Invention are, are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. An article for cleansing body surfaces, said article comprising:
a. an effervescent composition;
b. a first layer, said first layer is permeable to a liquid and comprises a web; and
c. a cleansing composition;
wherein said article has a Steady Flash Lather Volume of at least 250 ml.

2. The article of claim 1, further comprising a pouch comprising a second layer comprising at least two webs with said effervescent composition associated with said webs.

3. The article of claim 2, wherein said webs comprising said pouch are selected from the group consisting of nonwovens, formed films, films, composite material, laminates, wovens and combinations thereof.

4. The article of claim 1, further comprising a laminate comprising a second layer comprising at least two webs with said effervescent composition associated with said webs.

5. The article of claim 4, wherein said webs comprising said laminate are selected from the group consisting of nonwovens, formed films, films, composite material, wovens and combinations thereof.

6. The article of claim 2, said layers are bonded together about the perimeter of said article.

7. The article of claim 2, further comprising at least about 1.5% of said effervescent composition.

8. The article of claim 2, said effervescent composition further comprises an alkaline and an acidic material.

9. The article of claim 8, said alkaline is selected from the group consisting of anhydrous salts of carbonates and bicarbonates, alkaline peroxides, alkaline azides and mixtures thereof.

10. The article of claim 8, said alkaline is selected from the group consisting of sodium bicarbonate, potassium bicarbonate and mixtures thereof.

11. The article of claim 8, said acid material is selected from the group consisting of C₂ to C₂₀ organic mono- and poly-carboxylic acids, C₂ to C₂₀ organophosphorous acids, C₂ to C₂₀ organosulfur, toluene sulfonic acid, peroxides, lactones and mixtures thereof.

12. The article of claim 11, said C₂ to C₂₀ organic mono- and poly-carboxylic acids are selected from the group consisting of adipic, glutaric, succinic, tartaric, malic, maleic, lactic, salicylic, citric acids and mixtures thereof.

13. The article of claim 8, said effervescent composition is dry and comprises from about 1 weight percent to about 80 weight percent of the total weight of the effervescent composition of alkaline material.

14. The article of claim 8, said effervescent composition is dry and comprises from about 0.5 weight percent to about 80 weight percent of the total weight of the effervescent composition of acid material.

15. The article of claim 1, said cleansing composition is located on at least one surface of said web of said first layer.

16. The article of claim 1, said cleansing composition is from about 0.01 weight percent to about 1500 weight percent by weight of said article.

17. The article of claim 1, said cleansing composition is selected from the group consisting of anionic lathering surfactants, nonionic lathering surfactants, amphoteric lathering surfactants, and mixtures thereof.

18. The article of claim 17, said anionic lathering surfactant are selected from the group consisting of sarcosinates, isoeththionates, taurates phosphates, lactylates, glutamates and mixtures thereof.

19. The article of claim 1, said cleansing composition is a hard water tolerant surfactant that is selected from the group consisting of nonionic lathering surfactants, mid-chain branched surfactants, phosphates and mixtures thereof.

20. The article of claim 2, further comprising a third layer in communication with and juxtaposed to said second layer.

21. The article of claim 20, said third layer comprising a web further comprising a cleansing composition on at least one surface of said third layer.

22. The article of claim 20, further comprising a fourth layer in communication with and juxtaposed to said third layer wherein said fourth layer comprises high loft material which exhibits a density of from about 0.00005 g/cm3 to about 0.1 g/cm3 and a thickness of from about 0.1 mm to about 30 mm.

23. The article of claim 22, wherein said high loft material of said fourth layer comprises a cleansing composition associated with said fourth layer.

24. An article for cleansing body surfaces, said article comprising:
a. an effervescent composition;
b. a first layer, said first layer is permeable to a liquid and comprises a web; and
c. a cleansing composition;
wherein said article has a Steady Total Lather Volume of at least 850 ml.

25. The article of claim 24, further comprising a pouch comprising a second layer comprising at least two webs with said effervescent composition associated with said webs.

26. The article of claim 25, further comprising a third layer in communication with and juxtaposed to said second layer.

27. The article of claim 26, said third layer comprising a web further comprising a cleansing composition on at least one surface of said third layer.

28. The article of claim 26, further comprising a fourth layer in communication with and juxtaposed to said third layer wherein said fourth layer comprises high loft material which exhibits a density of from about 0.00005 g/cm3 to about 0.1 g/cm3 and an thickness of from about 0.1 mm to about 30 mm.

29. The article of claim 28, wherein said high loft material of said fourth layer comprises a cleansing composition associated with said fourth layer.

30. An article for cleansing body surfaces, said article comprising:
a. an effervescent composition;
b. a first layer, said first layer is permeable to a liquid and comprises a web; and
c. a cleansing composition;
wherein said article has a Rinsability Percent of greater than about 50%.

31. The article of claim 30, further comprising a pouch comprising a second layer comprising at least two webs with said effervescent composition associated with said webs.

32. The article of claim 31, further comprising a third layer in communication with and juxtaposed to said second layer.

33. The article of claim 32, said third layer comprising a web further comprising a cleansing composition on at least one surface of said third layer.

34. The article of claim 32, further comprising a fourth layer in communication with and juxtaposed to said third layer wherein said fourth layer comprises high loft material which exhibits a density of from about 0.00005 g/cm3 to about 0.1 g/cm3 and a thickness of from about 0.1 mm to about 30 mm.

35. The article of claim 34, wherein said high loft material of said fourth layer comprises a cleansing composition associated with said fourth layer.

36. A kit for the use of an article for cleansing body surfaces, said kit comprising:
a) an article for cleansing body surfaces, the article comprising:
1. an effervescent composition;
2. a first layer, said first layer is permeable to a liquid and comprises a web;
3. a cleansing composition; and
b) a package for storage of a plurality of said articles, said package having a removable seal and a closure, wherein opening the closure and removing said seal, a desired number of cleansing articles are removed from said package wherein the closure is closed to store the remaining non-selected articles in the package;
wherein said article has a Steady Flash Lather Volume of at least 250 ml, a Steady Total Lather Volume of at least 850 ml, a Rinsability Percent of greater than about 50%.

37. The kit according to claim 36 wherein the package comprises a hinged closure and said package comprises vents to allow escape of any residual gas from within the sealed package.

38. A kit for the use of an article for cleansing body surfaces, said kit comprising:
a) an article for cleansing body surfaces, the article comprising:
1. an effervescent composition;
2. a first layer, said first layer is permeable to a liquid and comprises a web;
3. a pouch comprising a second layer comprising at least two webs with said effervescent composition associated with said webs;
4. a third layer in communication with and juxtaposed to said second layer;
5. a fourth layer in communication with and juxtaposed to said third layer;
6. a cleansing composition; and
b) a package for storage of a plurality of said articles, said package having a removable seal and a closure, wherein opening the closure and removing said seal, a desired number of cleansing articles are removed from said package wherein the closure is closed to store the remaining non-selected articles in the package;
wherein said article has a Steady Flash Lather Volume of at least 250 ml, a Steady Total Lather Volume of at least 850 ml, a Rinsability Percent of greater than about 50%.

39. The kit according to claim 38 wherein the package comprises a hinged closure and said package comprises vents to allow escape of any residual gas from within the sealed package.

40. A kit for the use of an article for cleansing body surfaces, said kit comprising:
a) an article for cleansing body surfaces, the article comprising:
1. an effervescent composition;
2. a first layer, said first layer is permeable to a liquid and comprises a web;
3. a laminate comprising a second layer comprising at least two webs with said effervescent composition associated with said webs;
4. a third layer in communication with and juxtaposed to said second layer;
5. a fourth layer in communication with and juxtaposed to said third layer;
6. a cleansing composition; and
b) a package for storage of a plurality of articles, said package having a removable seal and a closure, wherein opening the closure and removing said seal, a desired number of cleansing articles are removed from said package wherein the closure is closed to store the remaining non-selected articles in the package;
wherein said article has a Steady Flash Lather Volume of at least 250 ml, a Steady Total Lather Volume of at least 850 ml, a Rinsability Percent of greater than about 50%.

41. The kit according to claim 40 wherein the package comprises a hinged closure and said package comprises vents to allow escape of any residual gas from within the sealed package.
